(19) United States
(12) Patent Application Publication
(10) Pub. No.: US 2010/0036277 A1
(43) Pub. Date: Feb. 11, 2010

(54) ANIMAL TEMPERATURE MONITOR AND MONITORING METHOD

(76) Inventor: John Austin, Narrandera (AU)

Correspondence Address: KIRK HAHN, 14431 HOLT AVE, SANTA ANA, CA, 92705, US

(21) Appl. No.: 12/307,722

(22) PCT Filed: Jul. 6, 2003
(86) PCT No.: PCT/AU07/00932

§ 371 Date: Oct. 17, 2009

(30) Foreign Application Priority Data
Jul. 6, 2006 (AU) 2006903660

(51) Int. Cl. A61B 5/01; A01K 29/00; A61B 5/11

(52) U.S. Cl. 600/549; 119/859; 600/595

(57) ABSTRACT

The invention provides apparatus 2, 12 for determining a condition-related temperature of an animal 1 having a tail 3. The apparatus comprises a temperature sensor 5 and means for determining and providing as an output a condition-related temperature of the animal from an output signal of the temperature sensor. The temperature sensor (or a component 16 bearing the temperature sensor) is secured to the animal so that the temperature sensor is positioned between the tail and left and right buttocks 13, 14 of the animal when the tail is in a specified normal position. The apparatus can be adapted for monitoring of condition-related temperature of several animals, and for remote monitoring.

### FIELD OF THE INVENTION

The invention here described relates to apparatus and methods for monitoring the temperature of animals, as a way of monitoring their wellbeing.

### BACKGROUND ART

It is an increasingly important requirement in the management, training and stabling of valuable animals to monitor their health. For example, the consequences of a racehorse or stud horse falling ill, or failing to recover quickly after doing so, can be of great financial importance. It is therefore widely desired to have ways to monitor the health of animals on a regular, or even continuous basis. Apart from horses, there is also interest in monitoring the health of other important animals, such as cattle and pigs.

Against this, there is a need to contain the cost of monitoring animals' condition—even in horse stables dealing with racehorses, it is not always possible to provide sufficient staff to closely monitor substantial numbers of animals.

These pressures have led to the development of animal health monitoring systems that operate automatically, or nearly so, and that have some remote monitoring, data recording and alerting capabilities.

One important indicator of an animal's condition is temperature, whether measured orally, rectally or otherwise. For example a raised temperature is commonly a sign that investigation, at least, is desirable. Systems have been developed that allow various temperatures of animals to be monitored remotely. However to date an important area of difficulty has been the actual provision of a transducer output responsive to a suitable temperature on an animal. It is known to implant transducers, and to place them against the skin or in body cavities such as the vagina in various ways. However each method has its own potential problems—for example, implanting can be costly, and carries the risk of infection when the implanting is done. Vaginal or rectal positioning of a temperature transducer can be uncomfortable and worrying for the animal.

The present invention provides temperature monitoring apparatus and a method for its installation and use that can provide an output satisfactorily indicative of the rectal temperature of certain animals, in particular horses, yet that is easy to put in place and to remove or replace as necessary, inexpensive, comfortable for the animal. The apparatus may have remote monitoring capabilities.

### DISCLOSURE OF THE INVENTION

According to the invention there is provided, in a first aspect, a method for determining a condition-indicating temperature of an animal having a tail, the method including the steps of:
(a) securing a temperature sensor to an animal having a tail so that when the tail is in a specified normal position close to the animal's buttocks the temperature sensor is positioned between the tail and left and right buttocks of the animal; and
(b) determining a condition-indicating temperature from an output signal of the temperature sensor.

When the tail is in the said normal position the sensor may be positioned at least partially within a cleft between the said buttocks of the animal.

Conveniently, the temperature sensor or a component bearing the temperature sensor may be secured to the tail of the animal.

The method may further include the steps of:
(a) also providing on the animal's tail a motion sensor or attitude sensor arranged to indicate movement of the tail from the specified normal position;
(b) monitoring an output signal of the motion or tilt sensor;
(c) accepting temperatures derived from output of the temperature sensor only when it can be inferred from output of the motion or attitude sensor that the tail has been at least approximately in the specified normal position long enough for the condition-related temperature to be adequately accurate.

Alternatively or additionally the method may include the step of analyzing temperatures or temperature-related signals derived from output of the temperature sensor to detect temperatures that are not representative of said condition-indicating temperature, and accepting only temperatures that are representative of said condition-indicating temperature.

The method may include the step of applying to a temperature as indicated by or estimated from output of the said temperature sensor an experimentally determined correlation between that temperature and a temperature as determined by placement of a thermometer in a cavity of the animal, preferably an ear, the anus, the rectum, the vagina or the mouth. (The temperature measured in the specified position between the buttocks and the tail may not be precisely identical to these but able to be related to any of them by an experimentally-determined correlation.)

There may be included the step of transmitting a temperature or a temperature-related output signal derived from the temperature sensor to a monitoring means remote from the temperature sensor. In fact, the monitoring means may be remote from the animal.

The said transmission may occur in response to an interrogation signal transmitted from a remote location. The signal may conveniently be transmitted by the remote monitoring means.

The temperature sensor may be one of a plurality of temperature sensors each fitted to a member of a plurality of animals and a temperature of each animal of said plurality is able to be determined and monitored. For example, several horses in a stable could be monitored at once.

Either the temperature or a temperature-related signal may be transmitted from a specific animal to a monitoring location in response to an interrogating signal that is transmitted from the monitoring location and that identifies the specific animal.

In a second aspect the invention provides apparatus for determining a condition-related temperature of an animal having a tail, comprising:
(a) a temperature sensor;
(b) securing means for securing to an animal having a tail the temperature sensor or a component bearing the temperature sensor so that the temperature sensor is positioned between the tail and left and right buttocks of the animal when the tail is in a specified normal position; and
(c) means for determining and providing as an output a condition-related temperature of the animal from an output signal of the temperature sensor.

The said temperature sensor or component bearing the temperature sensor may be secured to the animal so that the temperature sensor is positioned at least partially within a cleft between the said buttocks of the animal.

The securing means may be adapted to secure said temperature sensor or component bearing the temperature sensor to the tail of the animal.

The securing means may comprise a component bearing the temperature sensor and secured to said component at least one part that is adapted to be wrapped around the tail whereby to secure said component to the tail.

The securing means may
(a) define an opening through which the tail passes; and
(b) be tightenable about the tail to prevent slippage of the apparatus along the tail.

The apparatus may further comprise remote monitoring means remote from the animal and main transmission means for transmitting said condition-related temperature or a temperature-related signal derived from the temperature sensor from said animal to the remote monitoring means.

Said main transmission means may employ wireless transmission.

The apparatus may be adapted to transmit said condition-related temperature or temperature-related signal derived from the temperature sensor to said remote monitoring location in response to an interrogation signal transmitted from said remote monitoring means.

The apparatus may be adapted when transmitting to said remote monitoring means to transmit in addition to said condition-related temperature or temperature-related signal a signal individually identifying that part of the apparatus on the animal and thereby the animal. That is to say, where multiple animals are being monitored, the “on-animal” portion of the apparatus on each animal may have and transmit its own identifier, so that a particular animal with an abnormal reading can be identified from the identifier of the “on-animal” portion secured on it.

Information transmitted from the temperature sensor to the remote monitoring means may be transmitted by second transmission means to said main transmission means and thereafter from said main transmission means to said remote monitoring means. That is, there may be a portion of the apparatus secured to (or near) the tail and including the temperature sensor, and a portion secured elsewhere on the animal that has the transmission means for sending data to the monitoring means.

The transmission from said second transmission means to said main transmission means may be by wireless transmission. For example, the short-range “Bluetooth” standard may be used.

Said main transmission means may be secured to a portion of the animal other than the animal's tail.

The apparatus may include means for measuring on said animal a quantity other then the temperature derived from said temperature sensor and transmitting said quantity to said remote monitoring means. An example of this is given in the detailed description section hereof.

The apparatus may be characterized in that:
(a) said main transmission means and said temperature sensor are each members of sets of main transmission means and sensors respectively, each sensor and each main transmission means installed on one of a plurality of animals; and
(b) said remote monitoring means is adapted to receive transmissions of condition-related temperature or temperature-related signal from each of the main transmission means, whereby to monitor the plurality of animals.

The apparatus may further include a motion or attitude sensor secured to the tail of the animal and programmed means for inferring from output of said motion or attitude sensor whether the tail has been at least approximately in the specified normal position long enough for the condition-related temperature to be adequately accurate.

The apparatus may include programmed means for analyzing temperatures or temperature-related signals derived from output of the temperature sensor to detect temperatures that are not representative of said condition-indicating temperature, and accepting only temperatures that are representative of said condition-indicating temperature.

Further aspects and preferred features of the invention are set out in the remainder of this specification.

In this specification, including in the appended claims, the words “comprising” and “comprises” when used in relation to a set of elements integers features or steps are to be taken to indicate that the elements integers features or steps are present, but are not to be taken to preclude the possibility of other elements integers features or steps being present also.

Examples of the practice of the invention will now be described, by reference to the attached Figures.

### BRIEF DESCRIPTIONS OF THE VIEWS OF THE DRAWINGS

FIG. 1 is a side view of a horse fitted with a sensor package of an animal monitoring system according to the invention;

FIG. 2 is a perspective view of the sensor package shown in FIG. 1;

FIG. 3 is a cross-sectional view of the tail and a portion of the buttocks of the horse shown in FIG. 1, the section being taken at station “3-3” and as seen looking in the direction of arrow “A” (FIG. 2), with the sensor package of FIG. 2 now shown installed on the animal's tail;

FIG. 4 is a schematic block diagram of an animal monitoring system incorporating the sensor package as shown in FIGS. 1-3;

FIG. 5 is a side view of a horse fitted with a portion of an alternative animal monitoring system according to the invention;

FIG. 6 is a perspective view of the rear end of a horse fitted with another alternative embodiment of the invention.

### BEST MODES OF PERFORMING THE INVENTION

The following description is based on application of the invention to horses. However, it is to be explicitly understood that the invention is by no means limited in its application to horses.

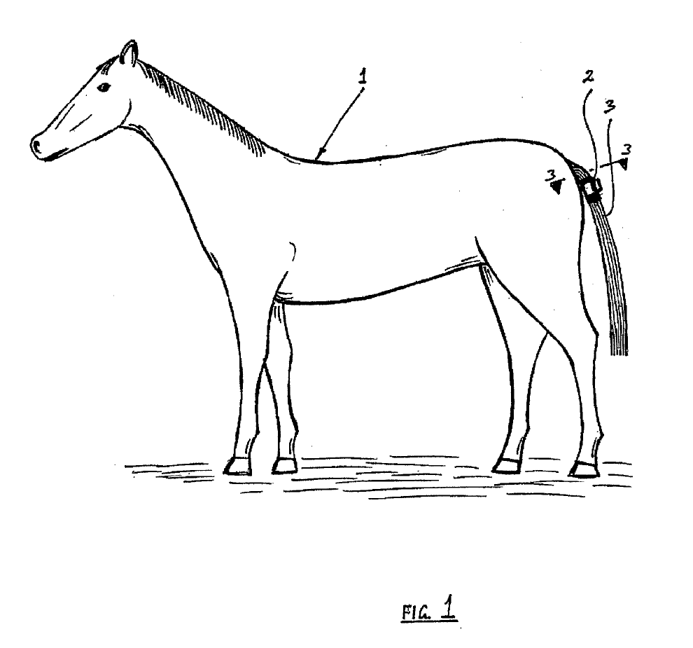

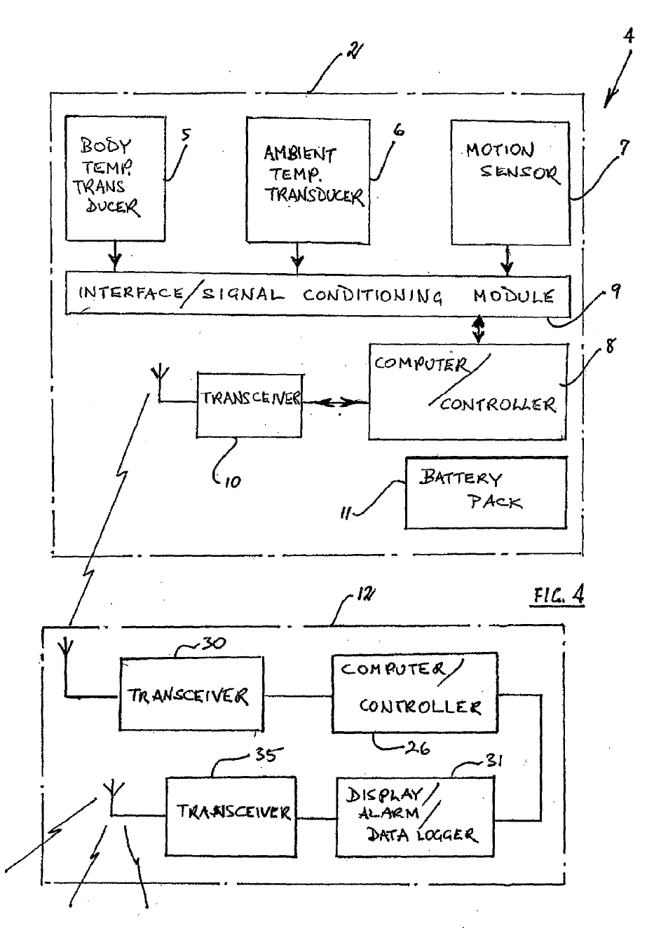

FIG. 1 shows a horse 1 fitted with a sensor package 2. Sensor package 2 is secured to tail 3 of horse 1 at an upper-end position on tail 3 adjacent to the horse's anus (not shown). FIG. 4 shows a schematic block diagram of a monitoring system 4 for animals which incorporates sensor package 2. The components incorporated in sensor package 2 are shown schematically in FIG. 4. These are a body temperature sensor (transducer) 5, an ambient temperature sensor 6, a motion sensor 7, a computer/controller module 8 based on a microprocessor, a module 9 for interfacing the sensors 5, 6 and 7 to the computer controller 8, a transceiver 10 and a battery pack 11. The transceiver 10 provides a wireless data link to a base station 12, which provides monitoring of the sensor package 2 and other sensor packages (not shown) in the monitoring system 4.

It is important to note that what is transmitted from sensor package 2 to package 12 to indicate temperature may be a quantity or datum directly representing temperature (eg a number that represents degrees centigrade) or a quantity or datum that must be in some way manipulated, for example in some form of computation such as multiplication by a voltage-to-temperature sensor calibration factor, to arrive at a temperature value in degrees. That is, derivation of temperature in actual degrees may take place in either package 2 or package 12. This general principle is equally applicable to the other embodiments described herein.

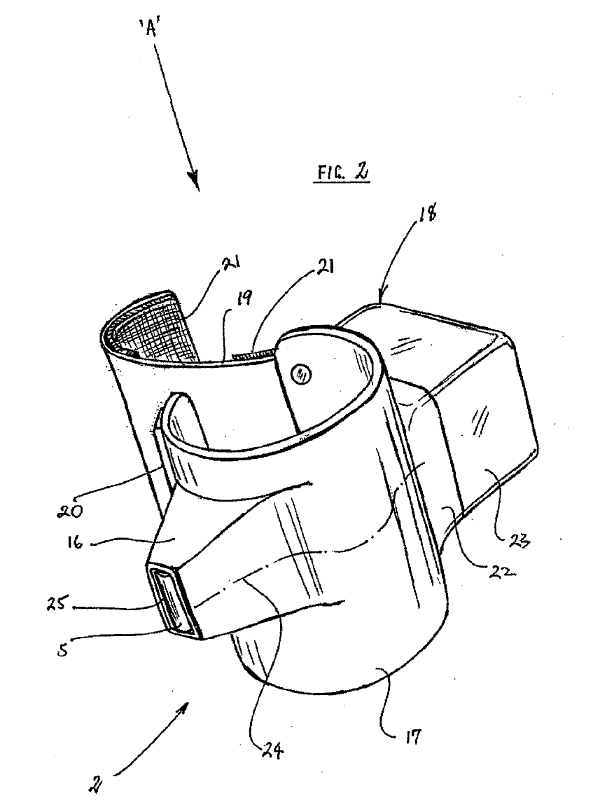

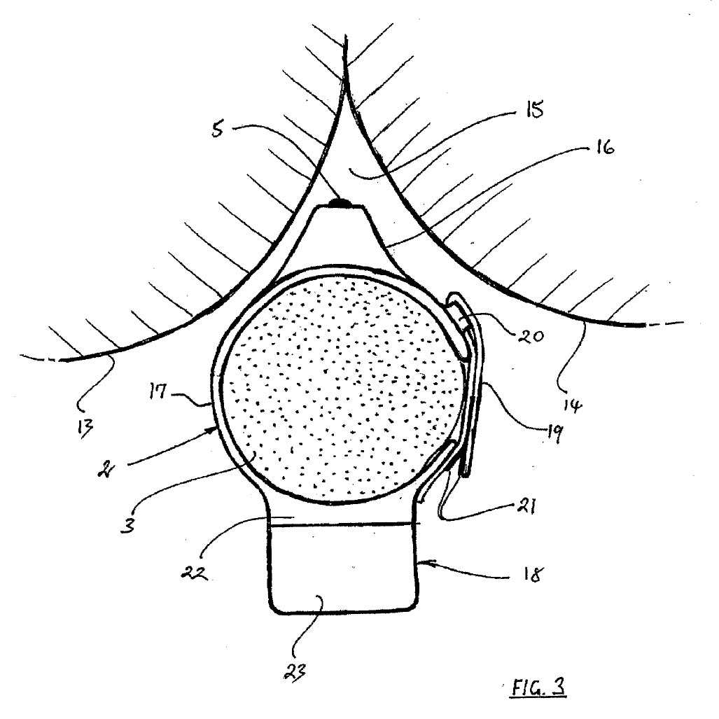

Referring to FIGS. 2 and 3, sensor package 2 is securely fitted to the tail 3 so that with tail 3 in its normal position, temperature sensor 5 is positioned as nearly as possible in the gap 15 between the left buttock 13 and right buttock 14 of horse 1 adjacent to the anus. Sensor 5 is supported in an open-fronted cavity 25 on a sensor support 16 of package 2 that is wedge-shaped and positioned to, in use, position sensor 5 neatly into gap 15 without excessively annoying horse 1. It has been found that a sensor measuring the temperature in gap 15, with tail 3 in its normal, downwardly hanging position, can give a temperature reading that is able to be related, usefully closely, to internal temperature of the horse 1, without any need for placement of a sensor internally or in a body cavity of the horse 1. In practice, depending on the nature of sensor 5, it may be found desirable for it to be positioned not in a shallow cavity like cavity 25, but wholly outside the boundaries of support 16. Determining this can be done by experiment with any proposed sensor 5 without the need for inventive activity.

The temperature measured by sensor 5 may be treated as a temperature indicative of animal condition in its own right, in the same way that rectal temperature, oral temperature or temperature within the ear can be. Alternatively, the system 4 may be programmed with a suitable algorithm to modify the temperature measured by sensor 5 to arrive at an estimate of one of the more widely recognized temperatures, such as for example rectal temperature. The algorithm to be used is itself not of the essence of this invention, but can depend on the nature of the sensor 5, the design of the support 16, and ambient temperature. For example, it may be found in particular circumstances (and without inventive activity) that simply adding a predetermined figure to the indicated temperature from sensor 5 gives a good estimate of rectal temperature.

The temperature sensors 5 and (where fitted) 6 may be of any type deemed suitable. Without any intention to preclude the possible use of other sensor types, sensors 5 and/or 6 could for example be of the Resistance Temperature Detector (RTD), Thermistor, Infrared or Integrated Circuit (IC) types. Integrated circuit type detectors are believed to lend themselves well to applications such as the present one due to their linearity, low power consumption and comparative ease of interfacing.

Sensor package 2 has a band 17 to which support 16 is secured along with a container 18 for the other components 6, 7, 8, 9 10 and 11. As best seen in FIG. 3, where tail 3 is shown in cross-section, band 17 is secured firmly around tail 3 at a position along its length that places support 16 and sensor 5 near the horse's anus. Band 3 includes on one side a strap 19 having hook-and-loop fastener halves 21 (eg of the type known as “Velcro”) that can be passed through a loop 20 and secured to itself to firmly secure sensor package 2 on tail 3 with support 16 in the required position. Band 17 may be formed from a somewhat flexible plastics material, for example, and support 16 may be integrally formed with band 17. Container 18 has a base part 22 and a cover 23 detachable for access and battery replacement, and the base part 22 may also be formed integrally with band 17. Suitable electrical connections 24 are provided to connect sensor 5 with the components in container 18, and may be run inside, outside or internally through band 17 in any suitable manner. It is convenient, and for horse 1 comfortable, that container 18 is positioned approximately diametrically opposite sensor support 16.

It is to be strongly emphasized that the particular form of construction of sensor package 2 described above and shown in FIGS. 1, 2, 3 and 4 is just one of many possible physical forms that will readily suggest themselves to a skilled person and that meet the following essential requirements:
(a) to hold the sensor 5 in gap 15 in use of sensor package 2 when tail 3 is in its normal position;
(b) to support and protect the other components 6, 7, 8, 9 10 and 11; and
(c) to be readily securable to tail 3 in the required orientation and position along its length.

Other suitable constructions that meet these requirements are also within the scope of the invention.

It will be recognized that if the horse 1 lifts, swings or otherwise moves its tail 3 so that sensor 5 is not positioned in gap 15, a temperature reading will be obtained that is not able to be simply related to the horse's body temperature. It is therefore necessary to ensure that such a condition can be recognized by the monitoring system 4. As one way of doing this, sensor package 2 is provided with a motion sensor 7. When the motion sensor 7 has an output signal indicating that horse 1 is swinging its tail or the like, system 4 can with suitable software indicate that a valid body temperature measurement is temporarily unavailable. This software could be run either in the computer/controller 8 in sensor package 2 or in the computer/controller in base station 12.

Motion sensor 7 could be an accelerometer of any suitable type, or a velocity sensor of any suitable type. Alternatively, although strictly not a “motion” sensor, a tilt sensor could be used, its signal indicating whether the tail 3 is in a suitable range of angles corresponding to normal tail position. Multi-axis sensors may be used if required.

Ambient temperature sensor 6 is provided so that a comparison can be made between its output and that of the body temperature sensor 5. The comparison can be useful as an adjunct to the output of the motion sensor 7 in determining whether the output of body temperature sensor 5 is genuine at a given time. It can also be used if necessary in adjusting the temperature output of sensor 5 to arrive at a definitive body temperature estimate. Further, ambient temperature is of interest in its own right, as a guide to the comfort level being experienced by the horse 1.

Although not shown in FIG. 4, the sensor package 2 may and preferably does include means for including in signals transmitted to base station 12 an identifier code that identifies the sensor package 2 itself. Provided a record is made of which animal 1 is fitted with which sensor package, the animal can be identified by the code even when multiple animals are being monitored by the monitoring system 4. As an example, the identifier code may be stored in read-only memory in sensor package 2. Preferably, the electronics of system 4 is digitally based, the signal transmitted to base station 12 comprising a sequence of digits including the identifier code and digitized readings from the sensors 5, 6 and 7. That scheme would apply if the software that uses the output of motion (or tilt) sensor 7 to determine the usability of the output of sensor 5 is in the computer/controller 26 of base station 12. However, if the output of the motion (or tilt) senor 7 is analyzed for this purpose by computer/controller 8, then the output of sensor 7 may be excluded from the string of digits transmitted.

FIG. 4 shows that both base station 12 and sensor package 2 contain transceivers (30 and 10 respectively) so that the communication between base station 12 and the (or each) sensor package 2 is two-way. This is the preferred arrangement and is provided so that the base station 12 can send interrogation signals to each of a number of sensor packages 2 in turn, receiving a response from each before moving on to the next. Each interrogation signal could for this purpose comprise a string of digits including the identifier code for one particular sensor package 2.

Where multiple sensor packages such as 2 are used, one for each of a group of animals to be monitored, the base station 12 could be arranged to “poll” each sensor package in turn. That is, a set of signals would be transmitted sequentially, each one containing an individual digital identifier for one of the sensor packages 2, and each sensor package would respond by transmitting a signal containing its temperature (or temperature-related) reading. The complete set of sensor packages would be interrogated by sequentially using each of their respective identifiers, and the whole cycle would then be repeated.

Alternatively, however, multiple sensor packages 2 could be arranged to simply transmit signal packets independently of each other and without any interrogation signal. Methods are well known in the art of radio-frequency identification (RFID) tagging for distinguishing and prioritizing amongst multiple signals from separate sources, and could be applied in a system such as system 4.

In base station 12, computer/controller 26 receives data from one or more sensor packages 2 via transceiver 30 and generates outputs in meaningful form for display by suitable display means 31 (eg computer screen), and/or for logging (by suitable recording means), and/or for alarming, where the estimated body temperature is outside a suitable band of allowable values. Base station 12 is also shown as having a second transceiver 35, which could provide a link to a remote location. As one example, transceiver 30 could provide a link to a remote location via a cellular telephone network.

Computer/controller 8 (or 26) may be programmed to ignore any reading from sensor 5 unless the motion (or tilt) sensor 7 has indicated that an acceptable tail 3 position has been maintained for a set period.

It is also possible to provide different programming in either or both computer/controllers 8 and 26 to suit different circumstances. For example, where sensor 7 is a tilt sensor, the output of sensor 7 could with suitable programming be monitored in its own right as an indicator of an animal lifting its tail in the manner known to occur when the animal is about to give birth. Although this application of tilt sensors is known, the present invention allows it to be made available as one of several options for animal monitoring.

It is not essential that wireless means be used for connecting the sensor package(s) to the base station in a monitoring system according to the invention, although wireless means do have the advantage that the animal(s) are able to move about without the hindrance of wires or cables. However, in a stable having a number of stalls for horses, for example, it may be perfectly acceptable to have a cable that connects the sensor package(s) 2 to the base station 12 since only limited animal movement is expected.

In an alternative to the embodiments set out above, the sensor(s) 7 discussed above are eliminated if required. Instead reliance is placed on recognizing that a rapid change of indicated temperature may, if the thermal capacity of the sensor 5 installation is sufficiently small, indicate a reading affected by tail movement or the like rather than a true change in the animal's temperature. Suitable programming can be provided to distinguish sensor outputs that are genuine from such affected outputs, and accept only the genuine outputs.

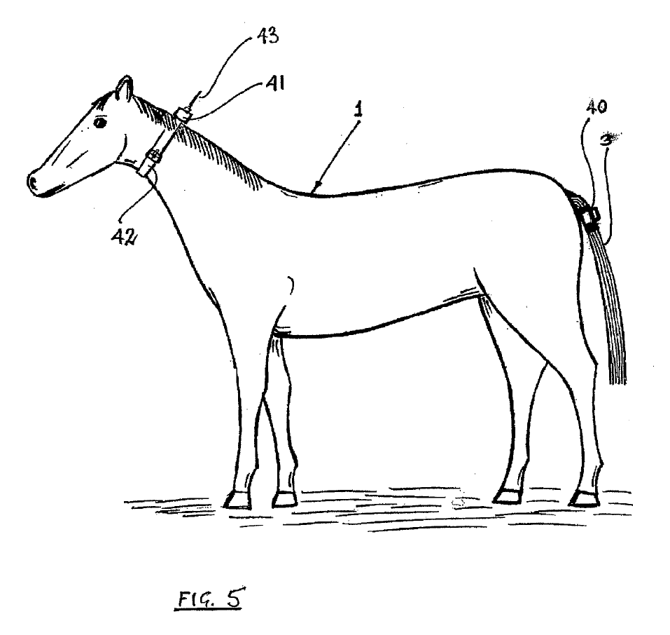

FIG. 5 shows an alternative embodiment of the invention in use on horse 1. Secured to the upper end of tail 3 of horse 1 is a sensor package 40 and secured on a neck collar 42 is a secondary package 41 with antenna 43. Sensor package 40 and secondary package 41 are in communication with each other but transmission of data between the horse 1 and a monitoring station (not shown, but equivalent to base station 12 of the system shown in FIG. 4) is by way of a data link between secondary package 41 and that monitoring station. This may be either by short-range wireless transmission means or alternatively by a cable connection (not shown). This embodiment has the ability to reduce any or all of the complexity, size, weight of the sensor package 40.

For example, the secondary package 41 could include a transceiver (not shown) with substantial range to enable communication with a base station (not shown), and a correspondingly high-capacity battery, while the sensor package 40 contains a transceiver (equivalent to transceiver 10 of the system shown in FIG. 4) only powerful enough to communicate with secondary package 41 and therefore smaller and lighter than for sensor package 2 and with a battery of lower capacity (hence size and/or weight). If packages 40 and 41 are connected by cable, sensor package 40 can have no transmitter at all and a correspondingly smaller battery.

The functionality and components of packages 40 and 41 could correspond to those of sensor package 2 and base station 12 as shown in FIG. 4, or be modified. For example, an ambient temperature sensor (not shown, but with the same purpose as item 6 in the system shown in FIG. 4) could be provided in secondary package 41 rather than sensor package 40. For further example, depending on the types of sensors used, their signal conditioning requirements, and the nature of the data link between packages 40 and 41, any computing/controlling function provided in sensor package 40 may be of lower capacity than that of computer controller 8 in the system shown in FIG. 4, or may even be eliminated altogether.

The data link between sensor package 40 and secondary package 41 could be, by way of non-limiting example, a link using the short-range “Bluetooth” standard.

Although the secondary package 41 has been shown as secured to the horse by way of a neck collar 42, any other suitable method of securing secondary package 41 to horse 1 could be used as required.

The embodiment shown in FIG. 5 also lends itself to the possibility (not shown) of having several temperature sensors on one animal, with each of them communicating with the secondary package 41. For example, if a horse has inflammation on a leg, reflected in a localized temperature rise, a temperature sensor could be placed under a bandage on the affected area to monitor it alongside the sensor placed under the tail and between the buttocks.

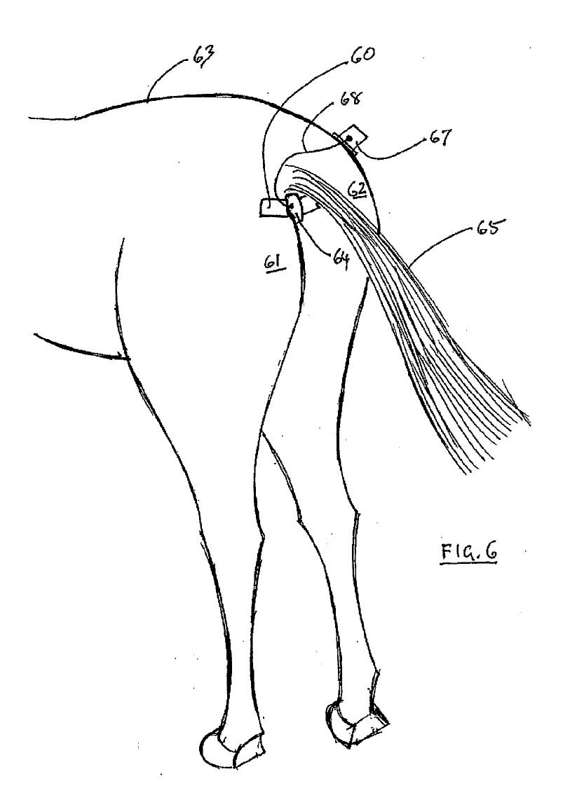

To place a temperature sensor in the space between an animal's buttocks and tail (as sensor 5 is shown between buttocks 13 and 14 and tail 3 of horse 1 in FIG. 3) an alternative arrangement is to secure the sensor or a component supporting the sensor to the body of the animal rather than to its tail. Such an embodiment is shown in FIG. 6. Adhesive tape 60 (or pad) is applied to the buttocks 61 and 62 of a horse 63, to secure a support 64 for a temperature sensor (not visible) under tail 65 and between buttocks 61 and 62. Tape 60 passes underneath, but close to, the tail 65. This approach has the disadvantage of potential fouling of the sensor when the animal defecates, although if the sensor support 64 is carefully positioned just above the anal opening this risk can be limited. The embodiments described above avoid the problem of fouling because the animal generally lifts its tail when defecating.

Separate means for detection of tail movement can be provided, for example by securing an accelerometer, tilt switch or the like (not shown) to the tail, but this can be less intrusive for the animal than a sensor package such as the sensor package 2 shown in FIG. 2. Alternatively, the approach set out above of avoiding the use of a motion sensor altogether may be used in this embodiment.

A sensor package 67 functionally equivalent to sensor packages 2 or 40 (except for the temperature sensor being external) can be adhesively or otherwise secured to the animal 63 in this embodiment and connected by a short cable 68 to the sensor support 64.

Although the description above has been based on apparatus for remote monitoring, it will be apparent to the skilled person that a readout could additionally or alternatively be provided on one of the parts secured to the animal.

Other variations, not exceeding the spirit or scope of the innovation, will readily suggest themselves to persons skilled in the art.

1-28. (canceled)

29. A method for determining a condition-indicating temperature of an animal having a tail, the method including the steps of:
(a) securing a temperature sensor to an animal having a tail so that when the tail is in a specified normal position close to the animal's buttocks the temperature sensor is positioned between the tail and left and right buttocks of the animal; and
(b) determining a condition-indicating temperature from an output signal of the temperature sensor.

30. The method of claim 29 wherein when the tail is in the said normal position the sensor is positioned at least partially within a cleft between the said buttocks of the animal.

31. The method of claim 29 wherein the temperature sensor or a component bearing the temperature sensor is secured to the tail of the animal.

32. The method of claim 29 further including the steps of:
(a) also providing on the animal's tail a motion sensor or attitude sensor arranged to indicate movement of the tail from the specified normal position;
(b) monitoring an output signal of the motion or tilt sensor; and
(c) accepting temperatures derived from output of the temperature sensor only when it can be inferred from output of the motion or attitude sensor that the tail has been at least approximately in the specified normal position long enough for the condition-related temperature to be adequately accurate.

33. The method of claim 29 including the step of analyzing temperatures or temperature-related signals derived from output of the temperature sensor to detect temperatures that are not representative of said condition-indicating temperature, and accepting only temperatures that are representative of said condition-indicating temperature.

34. The method of claim 29 including the step of applying to a temperature as indicated by or estimated from output of the said temperature sensor an experimentally determined correlation between that temperature and a temperature as determined by placement of a thermometer in a cavity of the animal, preferably an ear, the anus, the rectum, the vagina or the mouth.

35. The method of claim 29 including the step of transmitting a temperature or a temperature-related output signal derived from the temperature sensor to a monitoring means remote from the temperature sensor.

36. The method of claim 35 wherein the monitoring means is remote from the animal.

37. The method of claim 35 wherein the transmission occurs in response to an interrogation signal transmitted from a remote location.

38. The method of claim 29 wherein the temperature sensor is one of a plurality of temperature sensors each fitted to a member of a plurality of animals and a temperature of each animal of said plurality is able to be determined and monitored.

39. The method of claim 38 wherein either the temperature or a temperature-related signal is transmitted from a specific animal to a monitoring location in response to an interrogating signal that is transmitted from the monitoring location and that identifies the specific animal.

40. Apparatus for determining a condition-related temperature of an animal having a tail, comprising:
(a) a temperature sensor;
(b) securing means for securing to an animal having a tail the temperature sensor or a component bearing the temperature sensor so that the temperature sensor is positioned between the tail and left and right buttocks of the animal when the tail is in a specified normal position; and
(c) means for determining and providing as an output a condition-related temperature of the animal from an output signal of the temperature sensor.

41. The apparatus of claim 40 wherein when said temperature sensor or component bearing the temperature sensor is secured to the animal the temperature sensor is positioned at least partially within a cleft between the said buttocks of the animal.

42. The apparatus of claim 41 wherein the securing means is adapted to secure said temperature sensor or component bearing the temperature sensor to the tail of the animal.

43. The apparatus of claim 42 wherein the securing means comprises a component bearing the temperature sensor and secured to said component at least one part that is adapted to be wrapped around the tail whereby to secure said component to the tail.

44. The apparatus of claim 42 wherein the securing means
(a) defines an opening through which the tail passes; and
(b) is tightenable about the tail to prevent slippage of the apparatus along the tail.

45. The apparatus of claim 40 further comprising remote monitoring means remote from the animal and main transmission means for transmitting said condition-related temperature or a temperature-related signal derived from the temperature sensor from said animal to the remote monitoring means.

46. The apparatus of claim 45 wherein said main transmission means employs wireless transmission.

47. The apparatus of claim 45 adapted to transmit said condition-related temperature or temperature-related signal derived from the temperature sensor to said remote monitoring location in response to an interrogation signal transmitted from said remote monitoring means.

48. The apparatus of claim 45 adapted when transmitting to said remote monitoring means to transmit in addition to said condition-related temperature or temperature-related signal a signal individually identifying that part of the apparatus on the animal and thereby the animal.

49. The apparatus of claims 45 wherein information transmitted from the temperature sensor to the remote monitoring means is transmitted by second transmission means to said main transmission means and thereafter from said main transmission means to said remote monitoring means.

50. The apparatus of claim 45 wherein transmission from said second transmission means to said main transmission means is by wireless transmission.

51. The apparatus of claim 45 wherein said main transmission means is secured to a portion of the animal other than the animal's tail.

52. The apparatus of claim 45 including means for measuring on said animal a quantity other then the temperature derived from said temperature sensor and transmitting said quantity to said remote monitoring means.

53. The apparatus of claim 45 wherein:
(a) said main transmission means and said temperature sensor are each members of sets of main transmission means and sensors respectively, each sensor and each main transmission means installed on one of a plurality of animals; and
(b) said remote monitoring means is adapted to receive transmissions of condition-related temperature or temperature-related signal from each of the main transmission means, whereby to monitor the plurality of animals.

54. The apparatus of claim 40 further including a motion or attitude sensor secured to the tail of the animal and programmed means for inferring from output of said motion or attitude sensor whether the tail has been at least approximately in the specified normal position long enough for the condition-related temperature to be adequately accurate.

55. The apparatus of claim 40 including programmed means for analyzing temperatures or temperature-related signals derived from output of the temperature sensor to detect temperatures that are not representative of said condition-indicating temperature, and accepting only temperatures that are representative of said condition-indicating temperature.

56. The apparatus of claim 40 including programmed means for applying to a temperature as indicated by or estimated from output of the said temperature sensor an experimentally determined correlation between that temperature and a temperature as determined by placement of a thermometer in a cavity of the animal, preferably an ear, the anus, the rectum, the vagina or the mouth.
